(19) United States
(12) Patent Application Publication
(10) Pub. No.: US 2015/0073230 A1
(43) Pub. Date: Mar. 12, 2015

(54) CALCULATING BLOOD PRESSURE FROM ACOUSTIC AND OPTICAL SIGNALS

(71) Applicant: FUJITSU LIMITED, Kawasaki-shi (JP)

(72) Inventor: Stergios STERGIOU, East Palo Alto, CA (US)

(73) Assignee: FUJITSU LIMITED, Kawawaki-shi (JP)

(21) Appl. No.: 14/025,642

(22) Filed: Sep. 12, 2013

(51) Int. Cl. A61B 5/021

(52) U.S. Cl. CPC A61B 5/02108; A61B 5/02141
USPC 600/301

(57) ABSTRACT

A device configured to monitor blood pressure includes an audio sensor, an optical sensor, and a processing device. The audio sensor is configured to generate an audio data signal representing a heartbeat of a subject over time. The optical sensor is configured to generate an optical data signal representing blood volume within a blood vessel of the subject over time. The processing device is configured to calculate a pulse wave transit time (PWTT) based on the audio data signal and the optical data signal. The processing device is also configured to calculate a blood pressure of the subject based on the PWTT.

### FIELD

The embodiments discussed herein are related to calculating blood pressure from acoustic and optical signals.

### BACKGROUND

Unless otherwise indicated herein, the materials described herein are not prior art to the claims in the present application and are not admitted to be prior art by inclusion in this section.

Blood pressure (BP) is a biomarker with a rich body of research. One technique for obtaining continuous noninvasive BP (cniBP) involves the measurement of a parameter referred to as pulse wave transit time (PWTT). In a typical setting, an electrocardiograph (ECG) is used to detect the R peak of the QRS complex of a subject. Additionally, a photoplethysmograph (PPG) placed on the subject's finger is used to detect a blood volume peak in a blood vessel of the finger. PPGs generally operate by emitting light of certain frequencies and measuring absorption of the light through human tissue, such as a finger. The PWTT may be calculated based on the time delay between the R peak and the subsequent blood volume peak. The use of the ECG limits the deployability of blood pressure monitoring devices based on PWTT.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of an embodiment, a device configured to monitor blood pressure includes an audio sensor, an optical sensor, and a processing device. The audio sensor is configured to generate an audio data signal representing a heartbeat of a subject over time. The optical sensor is configured to generate an optical data signal representing blood volume within a blood vessel of the subject over time. The processing device is configured to calculate a pulse wave transit time (PWTT) based on the audio data signal and the optical data signal. The processing device is also configured to calculate a blood pressure of the subject based on the PWTT.

The object and advantages of the embodiments will be realized and achieved at least by the elements, features, and combinations particularly pointed out in the claims.

It is to be understood that both the foregoing general description and the following detailed description are exemplary and explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A is a block diagram of an example operating environment in which a noninvasive and continuous blood pressure monitoring device (hereinafter “device”) may be implemented;

FIG. 1B is a diagram of a specific implementation of the operating environment of FIG. 1A;

FIG. 2 is a block diagram of an example embodiment of the device of FIG. 1A;

FIG. 3 illustrates an example audio data signal representing a heartbeat of a subject over time;

FIG. 4 is a graph illustrating calculation of PWTT based on an audio data signal and an optical data signal; and

FIG. 5 shows an example flow diagram of a method of calculating blood pressure of a subject.

### DESCRIPTION OF EMBODIMENTS

The embodiments discussed herein are related to calculating blood pressure from acoustic and optical signals. For example, a blood pressure measurement based on PWTT may be calculated or otherwise derived from the acoustic and optical signals. As mentioned previously, the use of ECGs limits the deployability of blood pressure monitoring devices based on PWTT.

As already mentioned, PPGs operate by emitting light of certain frequencies and measuring its absorption through human tissue. PPG measurements have been successfully simulated via the use of smartphone cameras. However, standard smartphones are unable to calculate PWTT based solely on the simulated PPG measurements. Components external to such smartphones, such as an ECG chest strap and/or an ECG patch, may be provided to obtain an electrocardiogram so that PWTT may be calculated based on the simulated PPG measurements and the electrocardiogram.

It may also be feasible to obtain PWTT using two different PPG or pulse detectors. For instance, the camera of a smartphone may be used as one PPG to measure blood volume in a finger, while an external ear clip PPG may be used to measure blood volume in an ear. The measurements from the two PPGs may be used to calculate PWTT. Whether using a smartphone combined with an external ECG or a smartphone combined with an external ear clip PPG, the additional external components may add unnecessary complexity and/or inconvenience to the process of monitoring blood pressure for many people, including less technically savvy elderly persons.

Some embodiments described herein, however, include a smartphone or other device including an integrated audio sensor, such as a microphone, and an integrated optical sensor, such as a camera. The smartphone may use the microphone to generate an audio data signal representing a heartbeat of the subject over time. The smartphone may use the camera to generate an optical data signal representing blood volume within a blood vessel of the subject over time. The smartphone may then calculate PWTT based on the audio data signal and the optical data signal, and may calculate a blood pressure of the subject based on the PWTT. In comparison to other blood monitoring systems including a smartphone and one or more external components, embodiments described herein may be less complex and/or more convenient to use as the embodiments described herein may lack external on-body sensors such as PPG clips and ECG straps or patches.

Embodiments of the present invention will be explained with reference to the accompanying drawings.

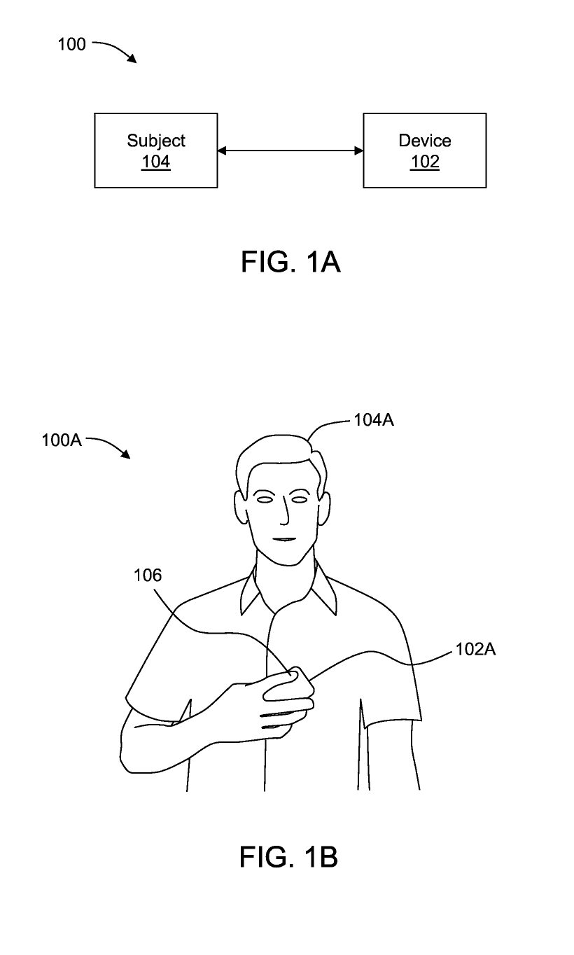

FIG. 1A is a block diagram of an example operating environment 100 in which a noninvasive and continuous blood pressure monitoring device (hereinafter “device”) 102 may be implemented, arranged in accordance with at least one embodiment described herein. The device 102 may be, for example, a mobile phone, a smartphone, a tablet computer, a laptop computer, a desktop computer, or other device generally including an audio sensor and an optical sensor, and/or including an interface to receive as input the output of such an audio sensor and/or optical sensor. The operating environment 100 may additionally include a subject 104. The term “subject” may be used generically herein to refer to virtually any living organism, such as a human, for which blood pressure measurements may be desired.

In general, the device 102 may be used to indirectly measure the blood pressure of the subject 104. More particularly, the device 102 may be configured to obtain a noninvasive biomarker, such as pulse wave transit time (PWTT), from measurements generated by the audio sensor and the optical sensor included in and/or communicatively coupled to the device 102, and to then calculate the blood pressure of the subject based on the PWTT. As used herein, PWTT may refer to the time it takes for an arterial pulse to propagate from a subject's heart to a specified point on the subject's body, such as a finger or ear lobe. A mean arterial blood pressure (MABP) of the subject 104 may then be calculated based on the PWTT.

In an example embodiment, the audio sensor is a microphone and the optical sensor is a camera, such as a charge-coupled device (CCD). The microphone and the optical sensor may be integrated in the device 102, which may be a smartphone or other device, as already mentioned. A microphone and at least one camera are standard on many smartphones.

In these and other embodiments, the microphone may measure the heartbeat of the subject 104. More particularly, the microphone may generate an audio data signal representing the heartbeat of the subject 104 over time by generating an electrical signal—e.g., the audio data signal—representing sound waves emitted by the heart of the subject 104 over time.

Alternately or additionally, the camera may generate a photoplethysmogram of a specified point on the body of the subject 104, such as a finger. More particularly, the camera may generate an optical data signal representing blood volume within a blood vessel at a specified point on the body of the subject 104 over time by generating an electrical signal—e.g., the optical data signal—representing light absorption of the finger over time.

Modifications may be made to the operating environment of FIG. 1A without departing from the scope of the described embodiments. For example, the operating environment 100 may additionally include one or more sensors, such as an audio sensor and/or an optical sensor, which are provided external to the device 102 and which are communicatively coupled to the device 102. Alternately or additionally, the operating environment may include a traditional cuff-based blood pressure monitor which may be used for calibrating the device 102, as described in more detail below.

FIG. 1B is a diagram of a specific implementation 100A of the operating environment 100 of FIG. 1A, arranged in accordance with at least one embodiment described herein. The specific implementation 100A of FIG. 1B includes a smartphone 102A, which is one example of the device 102 of FIG. 1A, and a person 104A, which is one example of the subject 104 of FIG. 1A.

As illustrated, to obtain a noninvasive and continuous blood pressure measurement, the person 104A may place the smartphone 102A on or against the chest of the person 104A such that a microphone or other audio sensor of the smartphone 102A is in a vicinity of the heart, e.g., close enough to the chest and/or the heart to measure sound waves emitted by the heart as it circulates blood through a circulatory system of the person 104A. The smartphone 102A may operate its microphone to generate an audio data signal representing the heartbeat of the person 104A over time by measuring sound waves emitted by the heart over time. In noisy environments, a noise-cancelling microphone may be utilized if such functionality is supported by the smartphone 102A.

Simultaneously, the person 104A may position a finger 106 against a camera or other optical sensor of the smartphone 102A. In some embodiments, the smartphone 102A may be positioned with a front of the smartphone 102A against the chest of the person 104A such that the finger 106 covers or is pressed against a back-facing camera of the smartphone 102A. Optionally, a flash—if present in the smartphone 102A—may be enabled to illuminate the finger 106. Whether based on ambient lighting or lighting from the flash, the smartphone 102A may operate its camera to generate an optical data signal representing blood volume within a blood vessel of the finger 106 over time by measuring light absorption of the finger 106 over time.

The smartphone 102A may then determine PWTT based on the audio data signal and the optical data signal. For instance, the smartphone 102A may detect S1 peaks in the audio data signal and corresponding blood volume peaks in the optical data signal. After time synchronizing the audio data signal and the optical data signal, if necessary, the smartphone 102A may calculate the PWTT as the time delay from an S1 peak in the audio data signal to a corresponding blood volume peak in the optical data signal. The PWTT may be calculated for each 51 peak and blood volume peak pair and the resulting PWTTs may be provided as instantaneous PWTTs. Alternately or additionally, multiple PWTTs may be averaged over a sliding time window to obtain an average PWTT. Based on an instantaneous PWTT, multiple instantaneous PWTTs, and/or an average PWTT, the smartphone 102A may calculate an instantaneous MABP, multiple instantaneous MABPs, or an average MABP, all of which may generically be referred to herein as simply the MABP.

The smartphone 102A may output the MABP to the person 104A through a suitable user interface of the smartphone 102A. For instance, the smartphone 102A may visually output the MABP to the person 104A by displaying it on a touchscreen of the smartphone 102A. Alternately or additionally, the smartphone 102A may audibly output the MABP to the person 104A via a speaker of the smartphone 102A. In these and other embodiments, the MABP may be saved on the smartphone 104A, used in health-related calculations, or reported to a healthcare provider, or the like or any combination thereof.

Modifications may be made to the specific implementation 100A of FIG. 1B without departing from the scope of the described embodiments. For example, the smartphone 102A may be communicatively coupled to an external photoplethysmograph sensor used to generate the optical data signal, rather than using the camera of the smartphone 102A. Alternately or additionally, the specific implementation 100A may include a traditional cuff-based blood pressure monitor which may be used for calibrating the device 102, as described in more detail below.

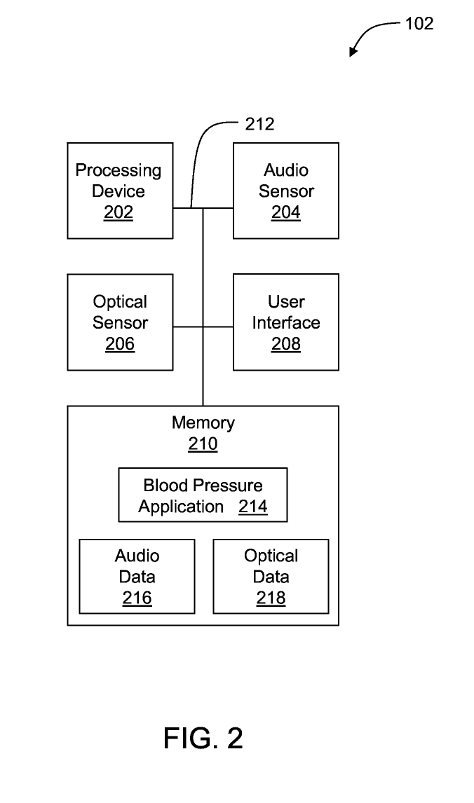

FIG. 2 is a block diagram of an example embodiment of the device 102 of FIG. 1A, arranged in accordance with at least one embodiment described herein. As already mentioned, the device 102 may be a mobile phone, a smartphone, a tablet computer, a laptop computer, a desktop computer, or other device generally including an audio sensor and an optical sensor, and/or including an interface to receive as input the output of such an audio sensor and/or optical sensor.

As illustrated, the device 102 includes a processing device 202, an audio sensor 204, and an optical sensor 206. Optionally, the device 102 may further include a user interface 208, a memory 210, and/or a communication bus 212. The device 102 will be described with combined reference to FIGS. 1A and 2.

The processing device 202 may be configured to execute computer instructions that cause the device 102 to perform the functions and operations described herein, such as operating an audio sensor to generate an audio data signal representing a heartbeat of the subject 104 over time, operating an optical sensor to generate an optical data signal representing blood volume with a blood vessel of the subject 104 over time, calculating a PWTT based on the audio data signal and the optical data signal, and calculating a blood pressure of the subject 104 based on the PWTT. The foregoing describes an example of a software implementation. Alternately or additionally, one or more of the functions and operations described herein may be implemented in hardware and/or firmware. The processing device 202 may include, but is not limited to, a processor, a microprocessor (g), a controller, a microcontroller (X), a central processing unit (CPU), a digital signal processor (DSP), any combination thereof, or other suitable processing device.

The audio sensor 204 may generally be configured to generate an audio data signal representing a heartbeat of the subject 104 over time. In some embodiments, the audio data signal may be an electrical signal generated as a measurement of sound waves emitted by the heart of the subject 104. In particular, as the sound waves generated by the beating heart arrive at the audio sensor 204, the audio sensor 204 may transduce the arriving sound waves to the electrical signal with a time-varying magnitude that corresponds to a time-varying magnitude of the arriving sound waves. The audio sensor 204 may include any suitable audio sensor such as, but not limited to, a carbon microphone, a piezoelectric transducer, a fiber optic microphone, a laser microphone, a liquid microphone, a MicroElectrical-Mechanical System (MEMS) microphone, or the like or any combination thereof.

The optical sensor 206 may generally be configured to generate an optical data signal representing blood volume within a blood vessel of the subject 104 over time. In some embodiments, the optical sensor 206 may operate as a photoplethysmograph. Accordingly, the optical data signal may be an electrical signal generated as a measurement of the blood volume within the blood vessel. In particular, during each heartbeat of the subject 104, the blood volume within the blood vessel may vary, resulting in a concomitant change in the absorption of light by the blood within the blood vessel. The optical sensor 206 may transduce light arriving at the optical sensor 206 to the electrical signal with a time-varying magnitude that corresponds to a time-varying intensity of the arriving light. The optical sensor 206 may be provided on one side of the finger, earlobe, or other part of the body of the subject 104 while a light source—such as ambient lighting—is provided on an opposite side in a transilluminated photoplethysmograph configuration. Alternately, the optical sensor 206 may be provided on the same side of the finger, earlobe, or other part of the body of the subject 104 as the light source—such as a flash of the device 102—in a back scattered photoplethysmograph configuration. The optical sensor 206 may include any suitable optical sensor such as, but not limited to, a CCD, a complementary metal-oxide-semiconductor (CMOS) active-pixel sensor (APS), or the like or any combination thereof.

The user interface 208 may be configured to receive input from and/or provide output to the subject 104. By way of example, the user interface 208 may be configured to receive input effective to control operation of the device 102, including starting and/or stopping noninvasive and continuous blood pressure monitoring by the device 102. As another example, the user interface 208 may be configured to display or otherwise output to the subject 104 the PWTT and/or the MABP of the subject 104. In these and other embodiments, the user interface 208 may include, but is not limited to, a keyboard, a mouse, a touchscreen or other touch input device, a voice input device, a speaker, or the like or any combination thereof.

The memory 210 may generally include a non-transitory computer-readable medium, such as random access memory (RAM) or other suitable volatile storage. Alternately or additionally, the memory 210 may include nonvolatile storage. More generally, the device 102 may include a non-transitory computer-readable medium such as, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory computer-readable medium. Computer instructions may be loaded into the memory 210 for execution by the processing device 202 to cause the processing device 202 to perform one or more of the operations described herein.

Alternately or additionally, the memory 210 may include a blood pressure application 214. The blood pressure application 214 may include the computer instructions that, when executed by the processing device 202, cause the processing device 202 to perform one or more of the operations described herein, such as the operations involved in noninvasive and continuous blood pressure monitoring, including calculating the PWTT and/or the MABP of the subject 104. In some embodiments, the blood pressure application 214 may be implemented as a software application designed to run on smartphones, tablet computers, and/or other mobile devices. Such software applications are often referred to as apps.

Data generated, received, and/or operated on during performance of the functions and operations described herein may be at least temporarily stored in the memory 210. For example, the audio data signal generated by the audio sensor 204 may be at least temporarily stored in the memory 210 as audio data 216. As another example, the optical data signal generated by the optical sensor 206 may be at least temporarily stored in the memory 210 as optical data 218.

The processing device 202, the audio sensor 204, the optical sensor 206, the user interface 208, and the memory 210 may be communicatively coupled via the communication bus 212. The communication bus 212 may include, but is not limited to, a memory bus, a storage interface bus, a bus/interface controller, an interface bus, or the like or any combination thereof.

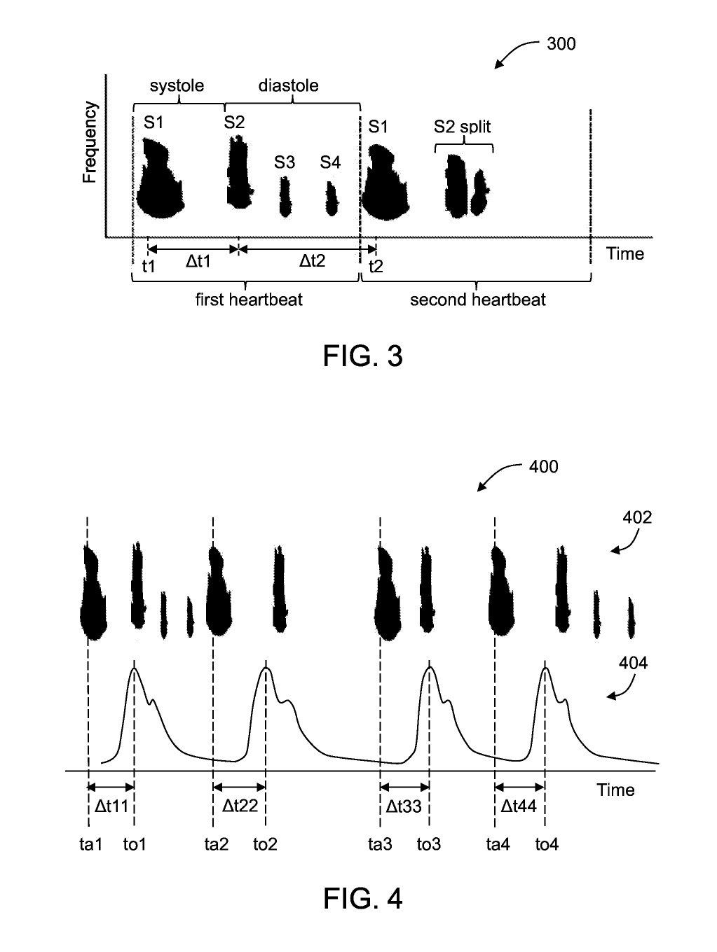

FIG. 3 illustrates an example audio data signal 300 representing a heartbeat of a subject over time, arranged in accordance with at least one embodiment described herein. The audio data signal 300 reflects the noises generated by the beating heart and the resulting flow of blood through it for a first and second heartbeat, although in other embodiments the audio data signal may include more heartbeats. In a healthy adult, there are two normal heart sounds often described as a lub and a dub that occur in sequence with each heartbeat. These sounds include a first heart tone S1 and a second heart tone S2 produced by, respectively, the closing of the atrioventricular (AV) valves (e.g., the mitral valve and the tricuspid valve) and the semilunar valves (e.g., the aortic valve and the pulmonary valve) of the heart. Signal peaks S1 and S2 corresponding to the first and second heart tones S1 and S2 are illustrated in the audio data signal 300.

A variety of other sounds may sometimes be present, including heart murmurs, adventitious sounds, and/or gallop rhythms S3 and S4. Signal peaks S3 and S4 corresponding to gallop rhythms S3 and S4 are illustrated for the first heartbeat of the audio data signal 300.

As illustrated in FIG. 3, the first heart tone S1 occurs at the beginning of ventricular systole, while the second heart tone S2 occurs at the beginning of ventricular diastole. The second heart tone S2 may sometimes be split, as illustrated for the second heartbeat of FIG. 3. Splitting of the second heart tone S2, also known as physiological split, normally occurs during inspiration. When present, gallop rhythms S3 and S4 may occur during ventricular diastole.

In some embodiments, the audio data signal 300 may be recorded for a predetermined amount of time and an average amplitude may be obtained to normalize the audio data signal. Alternately or additionally, the audio data signal 300 may be passed through a high-pass filter or other suitable filter such that signal peaks in the audio data signal 300 having amplitudes below a threshold amplitude are filtered from the audio data signal 300. In some embodiments, the threshold amplitude may be selected to pass signal peaks S1 and S2 and to filter out signal peaks S3 and S4 and/or signal peaks corresponding to other heart sounds.

The filtered audio data signal may be further processed to determine which of the S1 and S2 peaks remaining in the filtered audio data signal are S1 peaks, as the S1 peaks may be the peaks used for calculating the PWTT. In these and other embodiments, the time delay between each set of adjacent peaks may be calculated. The time delay may be calculated as an elapsed time from a peak amplitude within one peak to a peak amplitude within an adjacent peak. As illustrated in FIG. 3, a time delay Δt1 between the S1 peak and the S2 peak of the first heartbeat is shorter than a time delay Δt2 between the S2 peak of the first heartbeat and the S1 peak of the second heartbeat. The foregoing relationship generally holds true for every S1-S2-S1 sequence such that the time delay between each set of adjacent peaks may be used to determine which of the signal peaks in the filtered audio data signal are S1 peaks in view of the foregoing relationship. Where an S2 peak is split as in the second heartbeat of FIG. 3, the magnitude of the time delay may be so short as to easily identify a split S2 peak as such without incorrectly identifying one of the halves of the split S2 peak as being an S1 peak.

After the S1 peaks have been identified, a time at which each of the S1 peaks occurred may be returned for use in calculating the PWTT. For example, a time t1 may be returned for the S1 peak in the first heartbeat, and a time t2 may be returned for the S1 peak in the second heartbeat in FIG. 3.

FIG. 4 is a graph 400 illustrating the calculation of PWTT based on an audio data signal and an optical data signal, arranged in accordance with at least some embodiments described herein. The graph 400 includes an example audio data signal 402 representing a heartbeat of a subject over time and an example optical data signal 404 representing blood volume within a blood vessel of the subject over time. Each of the audio data signal 402 and the optical data signal 404 covers four heartbeats (not labeled). Moreover, the audio data signal 402 and the optical data signal 404 may be time synchronized.

As illustrated in FIG. 4, each of the heartbeats covered by the audio data signal 402 includes an S1 peak and an S2 peak (not labeled in FIG. 4). Moreover, some of the heartbeats covered by the audio data signal 402 may have S3 and S4 peaks (not labeled in FIG. 4), or peaks corresponding to additional heart sounds, while other heartbeats do not.

The audio data signal 402 may be filtered and the S1 peaks in the filtered audio data signal 402 may be identified as already described above to return times ta1, ta2, ta3, and ta4 at which the S1 peaks occur. Analogously, blood volume peaks in the optical data signal 404 may be identified to return times to1, to2, to3, and to4 at which the blood volume peaks occur.

A time delay, e.g., an elapsed time, may then be calculated between each S1 peak in the audio data signal 402 and the corresponding blood volume peak in the optical data signal 404. The time delays are identified in FIG. 4 as Δt11, Δt22, Δt33, and Δt44. Each of the time delays Δt11, Δt22, Δt33, and Δt44 are examples of instantaneous PWTTs. The PWTTs may be averaged over a sliding time window as previously described above.

The MABP may then be calculated based on the PWTT, e.g., based on at least one instantaneous PWTT and/or based on an average PWTT. A relationship between the MABP and the PWTT may be defined according to the Moens-Korteweg equation and the Hughes equation. The Moens-Korteweg equation and the Hughes equation are respectively provided below:

PWV²=(arterial_length/PWTT)²=Eh/ρ2r, and

E=E₀exp(αMABP).

In the Moens-Korteweg equation, PWV is pulse wave velocity, PWTT is the pulse wave transit time as already mentioned, arterial_length is an arterial length between a first location of a subject measured to generate the audio data signal and a second location of the subject measured to generate the optical data signal, E is Young's modulus of elasticity of a vessel wall of a blood vessel of the subject, h is a thickness of the vessel wall, ρ is a blood density of the subject, and r is a radius of the blood vessel. In the Hughes equation, E is the same as in the Moens-Korteweg equation, E₀ is a normalization factor, α is a constant approximately equal to 0.017 per millimeters mercury (mmHg), or 0.017 mmHg⁻¹, and MABP is the mean arterial blood pressure as already mentioned.

The Moens-Korteweg equation and the Hughes equation may be combined to relate PWTT and MABP to each other as follows:

(arterial_length/PWTT)²=[E₀exp(αMABP)]h/ρ2r.

Referring again to FIG. 1A, to calculate the MABP based on the PWTT in view of the Moens-Korteweg equation and the Hughes equation, the device 102 may first be calibrated against a standard arm-cuff based or other standard blood pressure monitor. In particular, the subject 104 may be simultaneously monitored by a standard blood pressure monitor and the device 102. Various parameters from the Moens-Korteweg equation and the Hughes equation, such as one or more of arterial_length, E, h, ρ, r, and E₀ may be determined by calibrating against blood pressure measurements generated by the standard blood pressure monitor. Determining the one or more parameters may include determining each of the one or more parameters individually or determining a single effective parameter that represents the one or more parameters in aggregate.

Additional details regarding the model represented by the Moens-Korteweg equation and the Hughes equation and the link between PWTT and blood pressure are provided in the following publications, each of which is incorporated herein by reference: D. J. Hughes et al., “Measurements of Young's modulus of elasticity of the canine aorta with ultrasound,” Ultrasonic Imaging 1 (1979): 356-367; H. H. Hardy et al., “On the pressure-volume relationship in circulatory elements,” Med. & Biol. Eng. & Comput. 20 (1982): 565-570; and Josep Solà et al., “Ambulatory Monitoring of the cardiovascular system: the role of Pulse Wave Velocity,” New Developments in Biomedical Engineering (2010): 391-424.

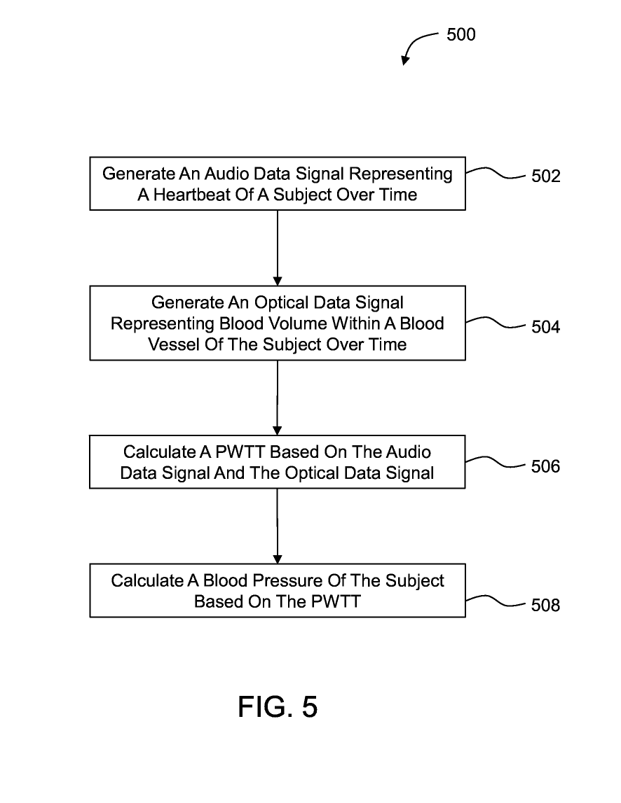

FIG. 5 shows an example flow diagram of a method 500 of calculating blood pressure of a subject, arranged in accordance with at least one embodiment described herein. The method 500 in some embodiments is performed by a device such as the device 102 of FIGS. 1A and 2 or the device 102A of FIG. 1B. Although illustrated as discrete blocks, various blocks may be divided into additional blocks, combined into fewer blocks, or eliminated, depending on the desired implementation.

The method 500 may begin at block 502 in which an audio data signal may be generated representing a heartbeat of a subject over time. The audio data signal may be generated by the audio sensor 204 of the device 102 of FIG. 2, for example. Generating the audio data signal representing the heartbeat of the subject over time may include using a microphone integrated in a smartphone positioned proximate a chest of the subject to generate an electrical signal representing sound waves emitted by a heart of the subject over time, where the electrical signal is the audio data signal.

At block 504, an optical data signal may be generated representing blood volume within a blood vessel of the subject over time. The optical data signal may be generated by the optical sensor 206 of the device 102 of FIG. 2, for example. Generating the optical data signal representing blood volume within the blood vessel of the subject over time may include using a camera integrated in a smartphone and against which the subject has positioned a finger as a photoplethysmograph to generate an electrical signal representing light absorption of the finger over time, where the electrical signal is the optical data signal.

At block 506, a PWTT may be calculated based on the audio data signal and the optical data signal. Calculating the PWTT based on the audio data signal and the optical data signal may include detecting a peak in the audio data signal, detecting a peak in the optical data signal, and calculating a time delay between a first time at which the peak in the audio data signal occurs and a second time at which the peak in the optical data signal occurs, where the PWTT is equal to the time delay. The foregoing aspects of calculating the PWTT are described in more detail above with respect to FIGS. 3-4.

At block 508, a blood pressure of the subject may be calculated based on the PWTT. Calculating the blood pressure of the subject based on the PWTT may include calculating a mean arterial blood pressure (MABP) of the subject based on the PWTT, where a relationship between the MABP and the PWTT is defined according to the Moens-Korteweg equation and the Hughes equation as already described above.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

For example, the method 500 may further include, prior to detecting the peak in the audio data signal, filtering out signal peaks in the audio data signal having an amplitude below a threshold amplitude such that peaks in the filtered audio data signal include S1 and S2 peaks. In these and other embodiments, detecting the peak in the audio data signal may include determining which of the S1 and S2 peaks in the filtered audio data signal are S1 peaks, and detecting a peak amplitude of an S1 peak and the time at which the peak amplitude of the S1 peak occurs.

Alternately or additionally, the method 500 may further include determining one or more parameters used in each of the Moens-Korteweg equation and the Hughes equation by calibrating against blood pressure measurements generated by an arm-cuff based or other blood pressure monitor.

Some embodiments described herein include a non-transitory computer-readable medium having computer instructions stored thereon that are executable by a noninvasive and continuous blood pressure monitoring device to perform one or more of the operations included in the method 500 of FIG. 5, such as the operations illustrated by blocks 502, 504, 506, and/or 508 in FIG. 5, and/or variations thereof. The noninvasive and continuous blood pressure monitoring device may include the device 102 or 102A of FIGS. 1A-2 and the non-transitory computer-readable medium may include the memory 210 of FIG. 2, for example.

The embodiments described herein may include the use of a special purpose or general purpose computer including various computer hardware or software modules, as discussed in greater detail below.

Embodiments described herein may be implemented using computer-readable media for carrying or having computer-executable instructions or data structures stored thereon. Such computer-readable media may be any available media that may be accessed by a general purpose or special purpose computer. By way of example, and not limitation, such computer-readable media may include tangible computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general purpose or special purpose computer. Combinations of the above may also be included within the scope of computer-readable media.

Computer-executable instructions comprise, for example, instructions and data which cause a general purpose computer, special purpose computer, or special purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the terms “module” or “component” may refer to specific hardware implementations configured to perform the operations of the module or component and/or software objects or software routines that may be stored on and/or executed by general purpose hardware (e.g., computer-readable media, processing devices, etc.) of the computing system. In some embodiments, the different components, modules, engines, and services described herein may be implemented as objects or processes that execute on the computing system (e.g., as separate threads). While some of the system and methods described herein are generally described as being implemented in software (stored on and/or executed by general purpose hardware), specific hardware implementations or a combination of software and specific hardware implementations are also possible and contemplated. In this description, a “computing entity” may be any computing system as previously defined herein, or any module or combination of modulates running on a computing system.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A device configured to monitor blood pressure, the device comprising:
an audio sensor configured to generate an audio data signal representing a heartbeat of a subject over time;
an optical sensor configured to generate an optical data signal representing blood volume within a blood vessel of the subject over time; and
a processing device configured to:
calculate a pulse wave transit time (PWTT) based on the audio data signal and the optical data signal; and
calculate a blood pressure of the subject based on the PWTT.

2. The device of claim 1, wherein the processing device is configured to calculate the PWTT based on the audio data signal and the optical data signal by:
detecting a peak in the audio data signal;
detecting a peak in the optical data signal; and
calculating a time delay between a first time at which the peak in the audio data signal occurred and a second time at which the peak in the optical data signal occurred, wherein the PWTT is equal to the time delay.

3. The device of claim 2, wherein the processing device is further configured to, prior to detecting the peak in the audio data signal, filter out signal peaks in the audio data signal having an amplitude below a threshold amplitude such that signal peaks in the filtered audio data signal include S1 and S2 peaks, wherein detecting the peak in the audio data signal includes:
determining which of the S1 and S2 peaks in the filtered audio data signal are S1 peaks; and
detecting a peak amplitude of an S1 peak.

4. The device of claim 1, wherein the processing device is configured to calculate the blood pressure of the subject based on the PWTT by calculating a mean arterial blood pressure (MABP) of the subject based on the PWTT, and wherein a relationship between the MABP and the PWTT is defined according to PWV=arterial_length/PWTT=(Eh/ρ2r) and E=E₀ exp(αMABP), where:
PWV is pulse wave velocity;
arterial_length is an arterial length between a first location of the subject measured to generate the audio data signal and a second location of the subject measured to generate the optical data signal;
E is Young's modulus of elasticity of a vessel wall of a blood vessel of the subject;
h is a thickness of the vessel wall;
ρ is a blood density of the subject;
r is a radius of the blood vessel;
E₀ is a normalization factor; and
α is a constant approximately equal to 0.017 per millimeters mercury (mmHg).

5. The device of claim 1, wherein the device is a smartphone and the audio sensor is a microphone integrated in the smartphone such that the audio sensor is configured to generate the audio data signal representing the heartbeat of the subject over time by generating an electrical signal representing sound waves emitted by a heart of the subject over time while the microphone is positioned against a chest of the subject, wherein the audio data signal includes the electrical signal.

6. The device of claim 1, wherein the device is a smartphone and the optical sensor is a camera integrated in the smartphone such that the optical sensor is configured to generate the optical data signal representing blood volume within the blood vessel of the subject over time by generating an electrical signal representing light absorption of a finger of the subject over time while the finger is positioned against the camera, wherein the optical data signal includes the electrical signal.

7. A method of calculating blood pressure of a subject, the method comprising:
generating an audio data signal representing a heartbeat of a subject over time;
generating an optical data signal representing blood volume within a blood vessel of the subject over time;
calculating a pulse wave transit time (PWTT) based on the audio data signal and the optical data signal; and
calculating a blood pressure of the subject based on the PWTT.

8. The method of claim 7, wherein calculating the PWTT based on the audio data signal and the optical data signal comprises:
detecting a peak in the audio data signal;
detecting a peak in the optical data signal; and
calculating a time delay between a first time at which the peak in the audio data signal occurs and a second time at which the peak in the optical data signal occurs, wherein the PWTT is equal to the time delay.

9. The method of claim 8, further comprising, prior to detecting the peak in the audio data signal, filtering out signal peaks in the audio data signal having an amplitude below a threshold amplitude such that signal peaks in the filtered audio data signal include S1 and S2 peaks, wherein detecting the peak in the audio data signal includes:
determining which of the S1 and S2 peaks in the filtered audio data signal are S1 peaks; and
detecting a peak amplitude of an S1 peak.

10. The method of claim 7, wherein calculating the blood pressure of the subject based on the PWTT includes calculating a mean arterial blood pressure (MABP) of the subject based on the PWTT, and wherein a relationship between the MABP and the PWTT is defined according to the Moens-Korteweg equation and the Hughes equation.

11. The method of claim 10, further comprising determining one or more parameters used in the Moens-Korteweg equation and the Hughes equation by calibrating against blood pressure measurements generated by an arm-cuff based blood pressure monitor.

12. The method of claim 7, wherein generating the audio data signal representing the heartbeat of the subject over time comprises using a microphone integrated in a smartphone positioned against a chest of the subject to generate an electrical signal representing sound waves emitted by a heart of the subject over time, wherein the electrical signal is the audio data signal.

13. The method of claim 7, wherein generating the optical data signal representing blood volume within the blood vessel of the subject over time comprises using a camera integrated in a smartphone and against which the subject has positioned a finger as a photoplethysmograph to generate an electrical signal representing light absorption of the finger over time, wherein the electrical signal is the optical data signal.

14. A non-transitory computer-readable medium having computer instructions stored thereon that are executable by a device to perform operations comprising:
generating an audio data signal representing a heartbeat of a subject over time;
generating an optical data signal representing blood volume within a blood vessel of the subject over time;
calculating a pulse wave transit time (PWTT) based on the audio data signal and the optical data signal; and
calculating a blood pressure of the subject based on the PWTT.

15. The non-transitory computer-readable medium of claim 14, wherein calculating the PWTT based on the audio data signal and the optical data signal comprises:
detecting a peak in the audio data signal;
detecting a peak in the optical data signal; and
calculating a time delay between a first time at which the peak in the audio data signal occurs and a second time at which the peak in the optical data signal occurs, wherein the PWTT is equal to the time delay.

16. The non-transitory computer-readable medium of claim 15, wherein the operations further comprise, prior to detecting the peak in the audio data signal, filtering out signal peaks in the audio data signal having an amplitude below a threshold amplitude such that signal peaks in the filtered audio data signal include S1 and S2 peaks, wherein detecting the peak in the audio data signal includes:
determining which of the S1 and S2 peaks in the filtered audio data signal are S1 peaks; and
detecting a peak amplitude of an S1 peak.

17. The non-transitory computer-readable medium of claim 14, wherein calculating the blood pressure of the subject based on the PWTT includes calculating a mean arterial blood pressure (MABP) of the subject based on the PWTT, and wherein a relationship between the MABP and the PWTT is defined according to the Moens-Korteweg equation and the Hughes equation.

18. The non-transitory computer-readable medium of claim 17, wherein the operations further comprise determining one or more parameters used in the Moens-Korteweg equation and the Hughes equation by calibrating against blood pressure measurements generated by an arm-cuff based blood pressure monitor.

19. The non-transitory computer-readable medium of claim 14, wherein generating the audio data signal representing the heartbeat of the subject over time comprises using a microphone integrated in a smartphone positioned against a chest of the subject to generate an electrical signal representing sound waves emitted by a heart of the subject over time, wherein the electrical signal is the audio data signal.

20. The non-transitory computer-readable medium of claim 14, wherein generating the optical data signal representing blood volume within the blood vessel of the subject over time comprises using a camera integrated in a smartphone and against which the subject has positioned a finger as a photoplethysmograph to generate an electrical signal representing light absorption of the finger over time, wherein the electrical signal is the optical data signal.
